(19) United States
(12) Patent Application Publication
(10) Pub. No.: US 2023/0000495 A1
(43) Pub. Date: Jan. 5, 2023

(54) CIRCULAR STAPLING DEVICE WITH TISSUE GRASPING MEMBERS

(71) Applicant: Covidien LP, Mansfield, MA (US)

(72) Inventors: Osvaldo Barrera, Madison, CT (US); Andrew M. Miesse, Westbrook, CT (US); Dwight Bronson, Cheshire, CT (US)

(21) Appl. No.: 17/363,816

(22) Filed: Jun. 30, 2021

(51) Int. Cl. A61B 17/115; A61B 17/28

(52) U.S. Cl. CPC A61B 17/1155; A61B 17/28; A61B 2017/2808

(57) ABSTRACT

A circular stapling device includes a reload assembly having a housing, an annular staple cartridge, and grasping members. The housing and the staple cartridge define channels that receive the grasping members. The grasping members are movable through the channels between retracted and extended positions to grasp tissue and pull the tissue into the housing during an anastomosis procedure.

### FIELD

This disclosure generally relates to surgical stapling devices and, more particularly, to circular stapling devices for performing anastomosis procedures.

### BACKGROUND

Circular stapling devices for performing surgical procedures such as anastomoses are well known. In an anastomosis procedure, ends of two organ sections are joined with the circular stapling device. Typically, a circular stapling device includes a handle assembly, an elongated shaft or adapter assembly, a reload assembly including a staple cartridge, and an anvil assembly that is mountable to the adapter assembly and is movable in relation to the reload assembly between open and clamped positions. In use, opposed tissue end margins of the organ sections are clamped between an anvil head of the anvil assembly and the staple cartridge and the device is fired to drive an annular array of staples from the staple cartridge through the tissue end margins of the organ sections for deformation against the anvil head. An annular knife positioned within the reload assembly is advanced to core or remove organ tissue interior of the staples to clear an internal tubular passage of the organ sections.

Complications during anastomoses procedures may result in a need for further operation and/or permanent ostomy. One complication is anastomotic leakage. The risk of anastomotic leakage is multi-factorial and may be affected by patient comorbidities, chemotherapy, the presence of microbiome pathogens, and stapling technique. One cause of anastomotic leakage may be due to bilateral dog ear formation. Bilateral dog ear formation results when the tissue end margins are not drawn fully into the reload assembly prior to firing of the circular stapling device. Thus, when the circular stapling device is fired to join the end margins of the organ sections, ends of one or both end margins of the organ sections are stapled in a position that resembles dog ears (FIG. 1).

A continuing need exists for a circular stapling device that can form an anastomosis without forming dog ears.

### SUMMARY

This disclosure is directed to a circular stapling device that includes a reload assembly having a housing and a staple cartridge that define channels through the reload assembly. The channels receive grasping members that are movable within the channels between retracted and extended positions. The grasping members each have a distal portion that includes a tissue engaging member. The grasping members are movable from their extended positions back towards their retracted positions during an anastomosis procedure to pull tissue into the housing of the reload assembly.

One aspect of the disclosure is directed to a circular stapling device including an adapter assembly, a reload assembly, a grasping member, and an anvil assembly. The adapter assembly has a proximal portion and a distal portion and includes an anvil retainer. The reload assembly is supported on the distal portion of the adapter assembly and includes a staple cartridge and a housing that defines a cavity. The housing has a proximal portion and a distal portion. The staple cartridge is supported on the distal portion of the housing and has an annular configuration. The housing and the staple cartridge define at least one channel that extends from the proximal portion of the housing through the staple cartridge. The anvil retainer extends through the cavity defined by the housing. The grasping member is movable within the at least one channel between a retracted position and an extended position and includes a body having a proximal portion and a distal portion. The distal portion of the body of the grasping member includes a tissue engaging portion that extends from the at least one channel to a position distal of the staple cartridge when the grasping member is in the extended position. The anvil assembly is releasably coupled to the anvil retainer and is movable in relation to the staple cartridge between open and clamped positions.

Other aspects of the disclosure are directed to a reload assembly that includes a staple cartridge, a housing, and a grasping member. The staple cartridge has an annular configuration. The housing defines a cavity and has a proximal portion and a distal portion. The staple cartridge is supported on the distal portion of the housing. The housing and the staple cartridge define at least one channel that extends from the proximal portion of the housing through the staple cartridge. The grasping member is movable within the at least one channel between retracted and extended positions and includes a body having a proximal portion and a distal portion. The distal portion of the body of the grasping member includes a tissue engaging portion that extends from the at least one channel to a position distal of the staple cartridge when the grasping member is in the extended position.

Other aspects of this disclosure are directed to a reload including a staple cartridge, a housing, and first and second grasping members. The staple cartridge has an annular configuration and includes an inner periphery. The housing defines a cavity and has a proximal portion and a distal portion. The staple cartridge is supported on the distal portion of the housing. The housing and the staple cartridge define first and second diametrically disposed channels that extend from the proximal portion of the housing through the inner periphery of the staple cartridge. The first and second grasping members are movable within the first and second channels from retracted positions to extended positions. The first and second grasping members each include a body having a proximal portion and a distal portion. The distal portions of the bodies of the first and second grasping members include tissue engaging portions that extend from the first and second channels to positions distal of the staple cartridge when the first and second grasping members are in their extended positions.

In aspects of the disclosure, the at least one channel includes a first channel and a second channel, and the grasping member includes a first grasping member and a second grasping member.

In some aspects of the disclosure, the first grasping member is received within the first channel and the second grasping member is received within the second channel.

In certain aspects of the disclosure, the first and second channels are diametrically opposed to each other.

In aspects of the disclosure, the staple cartridge has an inner periphery and the first and second channels extend through the inner periphery of the staple cartridge.

In some aspects of the disclosure, the tissue engaging portion of each of the first and second grasping members includes a hook portion.

In certain aspects of the disclosure, the tissue engaging portion of each of the first and second grasping members includes first and second jaws that are movable in relation to each other between open and closed positions.

In aspects of the disclosure, the first jaw is pivotably coupled to the second jaw.

In some aspects of the disclosure, the proximal portion of each of the first and second grasping members includes a grip portion.

In certain aspects of the disclosure, the grip portion includes a loop.

In aspects of the disclosure, the body of the grasping member is formed of a resilient material.

In some aspects of the disclosure, the stapling device includes a handle assembly, and the proximal portion of the adapter assembly is coupled to the handle assembly.

Other features of the disclosure will be appreciated from the following description.

### BRIEF DESCRIPTION OF THE DRAWINGS

Various aspects of a circular stapling device according to aspects of the disclosure are described herein below with reference to the drawings, wherein:

FIG. 1 is a side perspective view of an anastomosis with dog ear formation;

FIG. 2 is a perspective view of a circular stapling device including a powered handle assembly according to aspects of the disclosure with an anvil assembly of the stapling device separated from an anvil retainer of the stapling device;

FIG. 3 is a side perspective view of a distal portion of the stapling device shown in FIG. 1 with the anvil assembly removed and grasping member separated from a reload assembly of the stapling device;

FIG. 4 is an enlarged view of the indicated area of detail shown in FIG. 3;

FIG. 5 is an enlarged view of the indicated area of detail shown in FIG. 3;

FIG. 6 is an enlarged view of an alternative version of the grasping member of the stapling device shown in FIG. 1;

FIG. 7 is a side perspective view of first and second tissue end margins of first and second organ sections prior to anastomosis of the organ sections;

FIG. 8 is a side perspective view of the reload assembly of the stapling device shown in FIG. 3 shown in phantom and positioned within the first tissue end margin of the first organ section with the grasping members in a retracted position;

FIG. 9 is an enlarged view of the indicated area of detail shown in FIG. 8;

FIG. 10 is a side view of the reload assembly of the stapling device shown in FIG. 8 shown in phantom and positioned within the first tissue end margin of the first organ section with the grasping members in extended positions;

FIG. 11 is a side view of the reload assembly of the stapling device shown in FIG. 8 shown in phantom and positioned within the first tissue end margin of the first organ section with the grasping members partially retracted and engaged with the first tissue end margin of the first organ section;

FIG. 12 is a side perspective view of the distal portion of the stapling device shown in FIG. 3 shown in phantom with the reload assembly positioned within the first tissue end margin of the first organ section and the anvil assembly coupled to the anvil retainer and positioned in the second tissue end margin of the second organ section;

FIG. 13 is a side perspective view of the first and second organ sections after the anastomosis procedure has been performed; and

FIG. 14 is a cross-sectional view taken along section line 14-14 of FIG. 13.

### DETAILED DESCRIPTION

The disclosed circular stapling device will now be described in detail with reference to the drawings in which like reference numerals designate identical or corresponding elements in each of the several views. However, it is to be understood that disclosed aspects of the circular stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

This disclosure is directed to a circular stapling device that includes a reload assembly having a housing, an annular staple cartridge, and grasping members. The housing and the staple cartridge define channels that receive the grasping members. The grasping members are movable through the channels between retracted and extended positions to grasp tissue and draw the tissue into the housing during an anastomosis procedure.

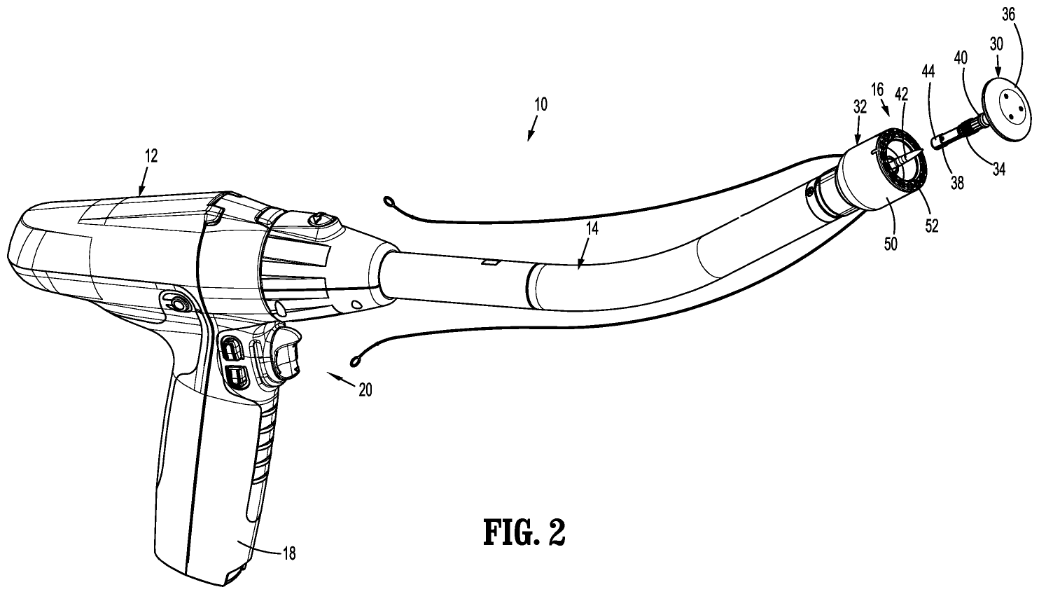

FIG. 2 illustrates a circular stapling device 10 shown generally as stapling device 10 that includes a handle assembly 12, an elongate body or adapter assembly 14 that extends from the handle assembly 12, and an end effector 16 that is coupled to the adapter assembly 14. The handle assembly 12 may be electrically powered and include a motor and associated gears and linkages to control operation of the stapling device 10. The handle assembly 12 includes a stationary grip portion 18 that supports a plurality of actuation buttons 20 that may be activated to control various functions of the stapling device 10, e.g., approximation of the end effector 16, firing of staples, and cutting of tissue. The stationary grip 18 may support a battery pack (not shown) that supplies power to the handle assembly 12. U.S. Pat. No. 10,327,779 discloses an exemplary powered circular stapling device.

Although the stapling device 10 is illustrated as a powered stapling device, it is envisioned that aspects of this disclosure could be incorporated into a manually powered stapling device. U.S. Pat. No. 10,022,126 (hereinafter “the '126 Patent”) discloses an exemplary manually actuated circular stapling device.

The end effector 16 of the stapling device 10 includes an anvil assembly 30 and a shell or reload assembly 32. The anvil assembly 30 includes an anvil shaft 34 and an anvil head 36. The anvil shaft 34 includes a proximal portion 38 and a distal portion 40 that supports the anvil head 36. In aspects of the disclosure, the anvil head 30 is pivotably coupled to the anvil shaft 34 and is movable from an operative position to a pivoted or tilted position. In the tilted position, the profile of the anvil head 36 is minimized to facilitate passage of the anvil assembly 30 through an organ section. The proximal portion 38 of the anvil shaft 34 is adapted to releasably engage an anvil retainer 42 of the stapling device 10. In aspects of the disclosure, the proximal portion 38 of the anvil shaft 34 includes resilient fingers 44 that define a longitudinal bore that receives the anvil retainer 42 of the stapling device 10 to releasably couple the anvil assembly 30 to the anvil retainer 42. For a detailed description of an anvil shaft and anvil retainer suitable for use with the stapling device 10, see the '126 Patent.

FIGS. 2-4 illustrate the reload assembly 32 which includes a housing 50 that defines a cylindrical cavity 50a and a staple cartridge 52 that is supported within the cylindrical cavity 50a on a distal portion of the housing 50. The staple cartridge 52 includes an annular body 54 that defines staple receiving slots 56 and includes a tissue contact surface 58. Each of the staple receiving slots 56 receives a staple 110 (FIG. 14). In aspects of the disclosure, the staple slots 56 are arranged in annular rows that are positioned about the annular body 54 of the staple cartridge 52. When the stapling device 10 (FIG. 2) is approximated by retracting the anvil retainer 42 into the housing 50 of the reload assembly 32, the anvil head 36 is moved to a clamped position in juxtaposed alignment with the tissue contact surface 58 of the staple cartridge 52. In some aspects of the disclosure, the reload assembly 32 is releasably coupled to the distal portion of the adapter assembly 14 and can be replaced after each firing of the stapling device 10 to facilitate reuse of the stapling device 10.

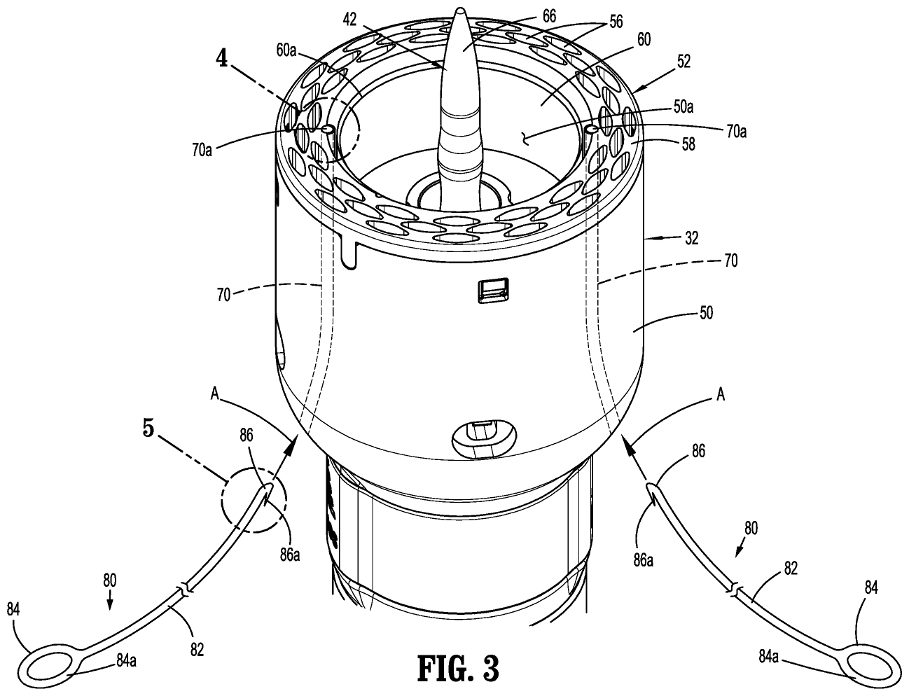

The reload assembly 32 includes an annular knife blade 60 that includes a cutting edge 60a. The knife blade 60 is movable within the cylindrical cavity 50a of the housing 50 and within an annulus of the staple cartridge 52 from a retracted position to an advanced position. In the retracted position of the knife blade 60, the cutting edge 60a of the knife blade 60 is recessed within the housing 50 (FIG. 3) of the reload assembly 32. In the advanced position of the knife blade 60, the cutting edge 60a of the knife blade 60 extends distal of the housing 50 of the reload assembly 32 and is engaged with the anvil head 36 of the anvil assembly 30.

Although not shown, the reload assembly 32 also includes a pusher that is movably supported within the housing 50 from a retracted position to an advanced position to eject the staples (not shown) from the staple cartridge 52. The pusher and the knife blade 60 can be coupled to each other and move in unison between their retracted and advanced positions. Alternately, the pusher can be moved between its retracted and advanced positions independently of the knife blade 60 to eject the staples from the staple cartridge 52, and the knife blade 60 can be subsequently moved between its advanced and retracted positions to cut tissue clamped between the anvil assembly 30 and the staple cartridge 52 of the reload assembly 32.

The anvil retainer 42 extends from the adapter assembly 14 through the cylindrical cavity 50a of the housing 50. In aspects of the disclosure, the anvil retainer 42 includes a distal trocar portion 66 that is received within the anvil shaft 34 of the anvil assembly 30 to releasably couple the anvil assembly 30 to the anvil retainer 42. The anvil retainer 42 is movable between advanced and retracted positions to move the anvil assembly 30 in relation to the staple cartridge 52 between the open and clamped positions.

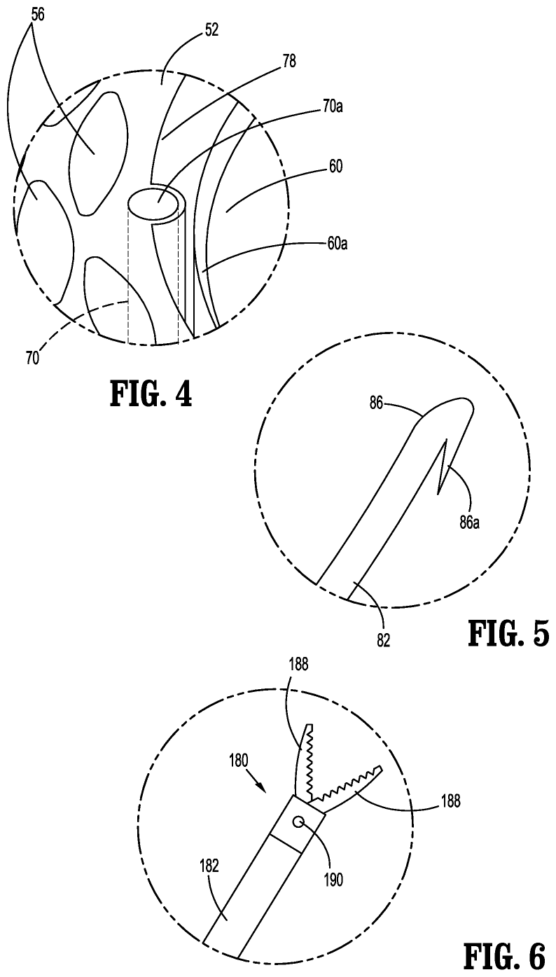

The housing 50 of the reload assembly 32 defines channels 70 that extend through the housing 50 from a proximal portion of the housing 50 to the distal portion of the housing 50. In aspects of the disclosure, the channels 70 communicate with channels 70a that extend through the staple cartridge 52. The channels 70 and 70a in the housing 50 and the staple cartridge 52 define a continuous channel through the reload assembly 32. In aspects of the disclosure, the channels 70a exit the staple cartridge 52 at diametrically opposed positions on an inner periphery or edge 78 of the staple cartridge 52 (FIG. 4) inwardly of the staple receiving slots 56. The channels 70, 70a of the housing 50 and the staple cartridge 52 are dimensioned to receive grasping members 80.

FIGS. 3 and 5 illustrate the grasping members 80. Each of the grasping members 80 includes an elongate body portion 82 that has a proximal portion and a distal portion. The proximal portion of each of the body portions 82 of the grasping members 80 includes a grip portion 84 that is configured for gripping by a clinician. The elongate body portion 82 of the grasping members 80 may have non-circular configurations, e.g., oval configurations, and the channels 70 may have corresponding non-circular configurations, to prevent rotation of the grasping members 80 within the channels 70. In aspects of the disclosure, the grip portion 84 of each of the grasping members 80 includes a loop 84a although other grip configurations are envisioned. In aspects of the disclosure, the grip portions 84 are dimensioned to prevent passage of the grip portions 84 of the grasping members 80 into the channels 70 of the housing 50. It is envisioned that the grasping members 80 have a length such that the grip portion 84 is accessible by a clinician adjacent the handle assembly 12.

The distal portion of the elongate body 82 of each of the grasping members 80 includes a tissue engaging portion 86 that is configured to grasp tissue positioned on the staple cartridge 52. In aspects of the disclosure, the tissue engaging portion 86 of each of the grasping members 80 includes a hook portion 86a. Alternately other tissue engaging configurations are envisioned. In aspects of the disclosure, the elongate body 82 is formed from a resilient material such as spring steel.

FIG. 6 illustrates an alternate version of the grasping member 80 shown generally as grasping member 180. The grasping member 180 includes an elongate body 182 that has a distal portion and a proximal portion. The proximal portion (not shown) has a grip portion and the distal portion of the elongate body 182 supports jaws 188. In aspects of the disclosure, the jaws 180 are coupled to the elongate body 182 by a pivot member 190 and are movable between open and closed positions.

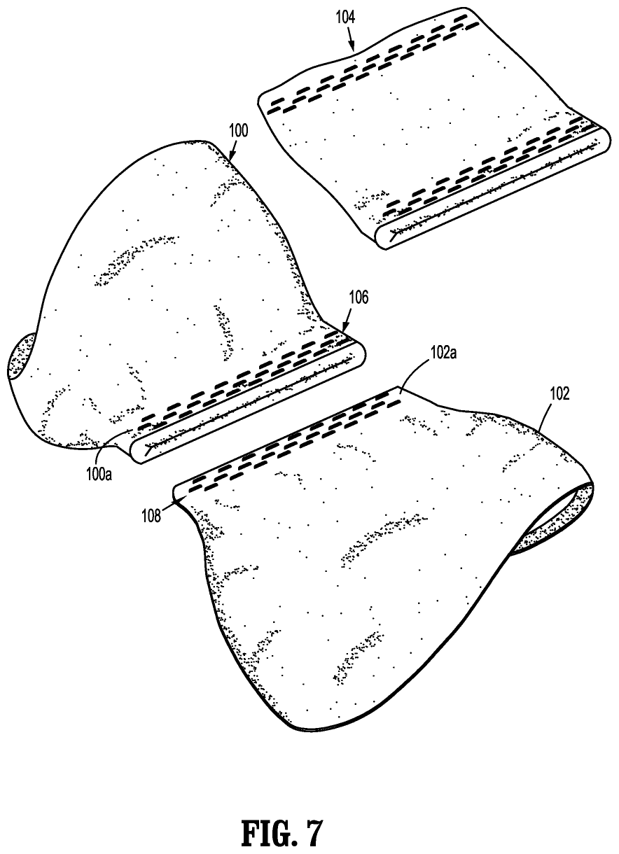

FIGS. 7-14 illustrate an anastomosis procedure being performed with the stapling device 10. FIG. 7 illustrates first and second tissue end margins 100a, 102a of first and second organ sections 100, 102 prior to anastomosis of the first and second organ sections 100, 102. Typically, prior to performance of anastomosis procedure, a diseased portion 104 of an organ, e.g., the colon, is resected from the organ using a linear stapling device (not shown). After the diseased portion 104 of the organ is resected, the first and second organ sections 100, 102 of the organ remain with staple lines 106, 108 formed in the end margins 100a, 102a of the first and second organ sections 100, 102.

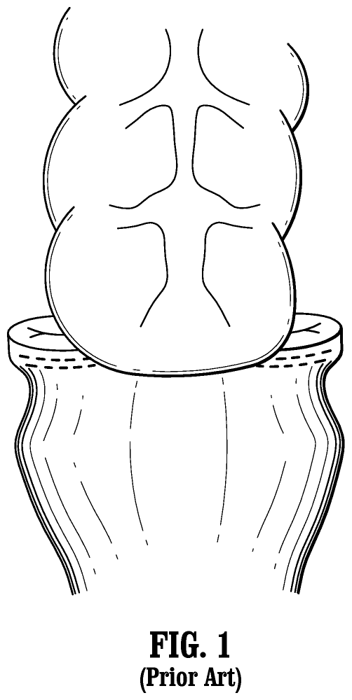

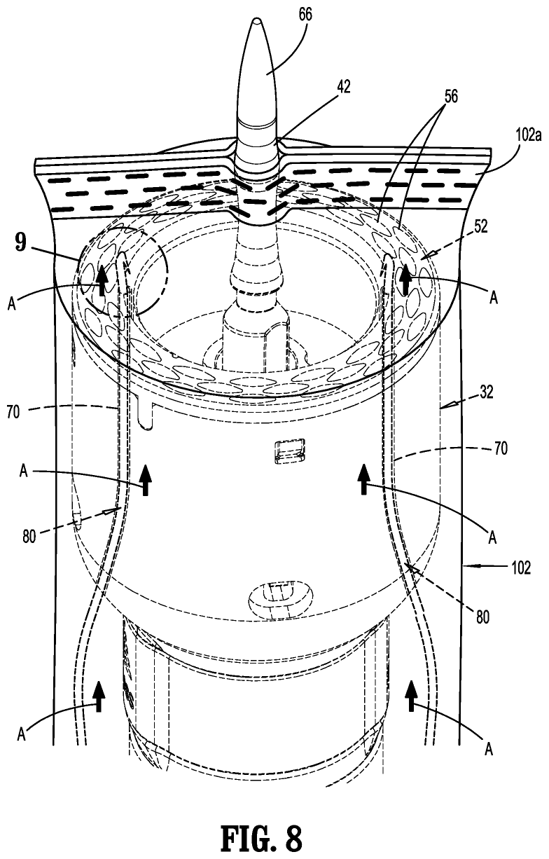

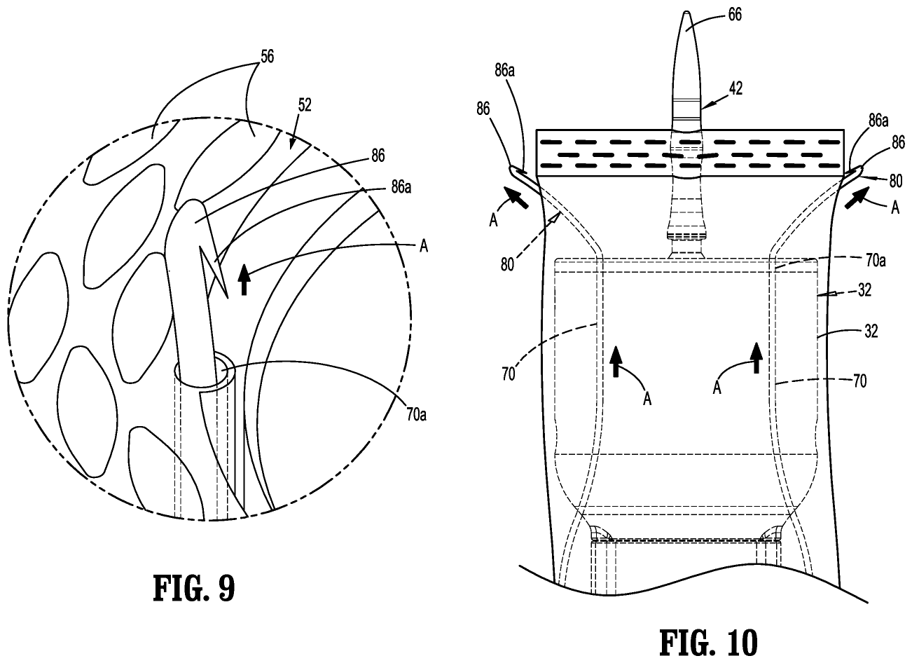

FIGS. 8-10 illustrate the distal portion of the stapling device 10 (FIG. 1) including the reload assembly 32 positioned within the second organ section 102 with the distal trocar portion 66 of the anvil retainer 42 inserted through the staple line 108 in the end margin 102a of the second organ section 102. Once the reload assembly 32 and the trocar portion 66 are positioned as shown, the grasping members 80 are inserted through the channels 70 formed in the housing 50 and the channels 70a formed the staple cartridge 52 in the direction of arrows “A” in FIGS. 3, and 8-10. As shown in FIGS. 9 and 10, the grasping members 80 are advanced through the channels 70, 70a until the tissue engaging portions 86 extend distally from the staple cartridge 52 and pass through the end margin 102a of the organ section 102. The grasping members 80 may be pre-shaped to extend outwardly from the channel 70 towards the tissue end margin 102a when the grasping members 80 exit the channels 70 (FIG. 10).

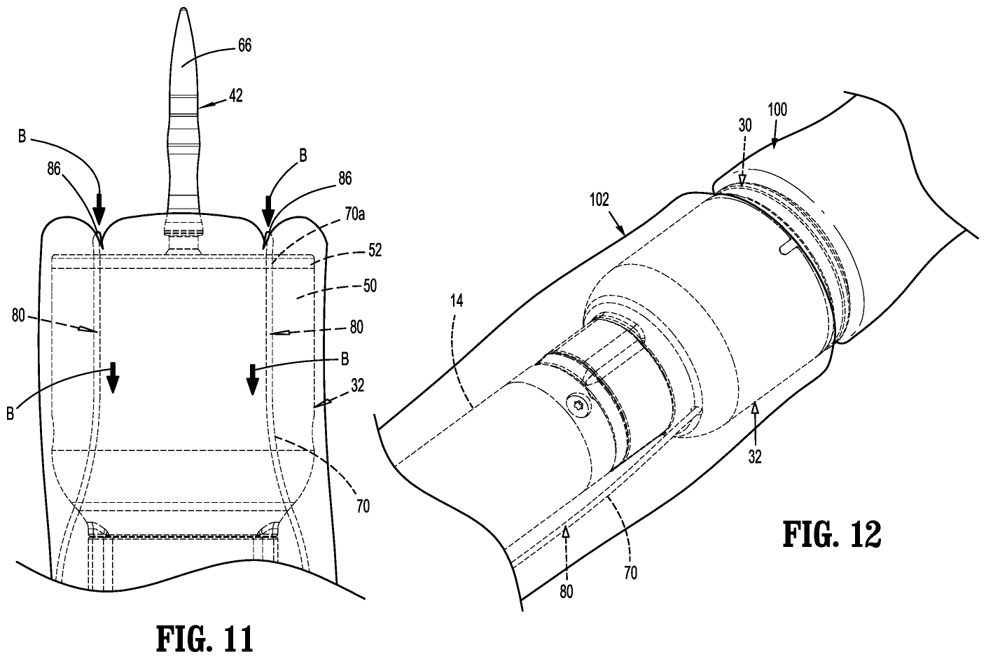

FIG. 11 illustrates the grasping members 80 as the grasping members 80 are retracted in the direction of arrows “B” to pull the end margin 102a of the organ section 102 into the housing 50 of the reload assembly 32. When the grasping members 80 are retracted in the direction of arrows “B”, the tissue engaging portions 86 of the grasping members 80 engage and pull the tissue towards the inner periphery of the staple cartridge 52.

FIG. 12 illustrates the distal portion of the stapling device 10 after the anvil assembly 30 is inserted into the first organ section 100 with the anvil shaft 34 of the anvil assembly 30 inserted through the staple line 106 in the end margin 100a of the first organ section 100. After the anvil shaft 34 is inserted through the staple line 106, the anvil shaft 34 is coupled to the anvil retainer 42 of the stapling device 10 (FIG. 1) and the anvil assembly 30 is moved in relation to the staple cartridge 52 to the clamped position. In the clamped position, the end margins 100a, 102a of the first and second organ sections 100, 102 are clamped between the anvil head 36 and the staple cartridge 52.

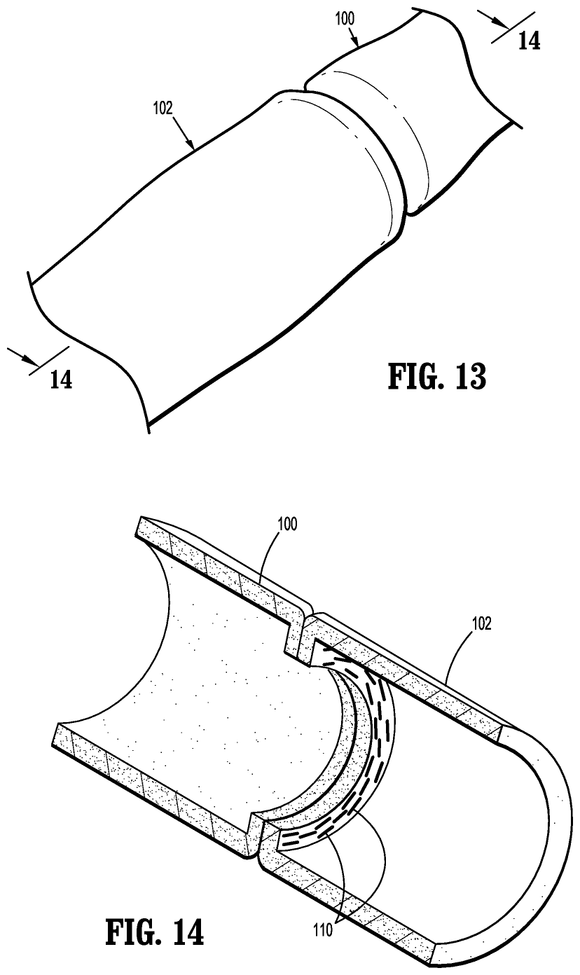

FIGS. 13 and 14 illustrate the organ sections 100, 102 after the anastomosis procedure has been performed and the stapling device 10 (FIG. 1) is removed from the organ sections 100, 102. Once the anvil assembly 30 is moved to the clamped position, the stapling device 10 (FIG. 1) is fired to eject the staples 110 (FIG. 14) through the organ sections 100, 102 to join the organ sections 100, 102. The annular knife blade 60 is also advanced to cut portions of the organ sections 100, 102 disposed within the housing 50 inwardly of the annular knife blade 60.

Persons skilled in the art will understand that the instruments and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A circular stapling device comprising:
an adapter assembly having a proximal portion and a distal portion, the adapter assembly including an anvil retainer;
a reload assembly supported on the distal portion of the adapter assembly and including a staple cartridge and a housing defining a cavity, the housing having a proximal portion and a distal portion, the staple cartridge supported on the distal portion of the housing and having an annular configuration, the housing and the staple cartridge defining at least one channel that extends from the proximal portion of the housing through the staple cartridge, the anvil retainer extending through the cavity defined by the housing;
a grasping member movable within the at least one channel between retracted and extended positions, the grasping member including a body having a proximal portion and a distal portion, the distal portion of the body of the grasping member including a tissue engaging portion, the tissue engaging portion extending from the at least one channel to a position distal of the staple cartridge when the grasping member is in the extended position;
an anvil assembly releasably coupled to the anvil retainer and movable in relation to the staple cartridge between open and clamped positions.

2. The circular stapling device of claim 1, wherein the at least one channel includes a first channel and a second channel, and the grasping member includes a first grasping member and a second grasping member, the first grasping member received within the first channel and the second grasping member received within the second channel.

3. The circular stapling device of claim 2, wherein the first and second channels are diametrically opposed to each other.

4. The circular stapling device of claim 3, wherein the staple cartridge has an inner periphery and the first and second channels extend through the inner periphery of the staple cartridge.

5. The circular stapling device of claim 2, wherein the tissue engaging portion of each of the first and second grasping members includes a hook portion.

6. The circular stapling device of claim 2, wherein the tissue engaging portion of each of the first and second grasping members includes first and second jaws, the first and second jaws movable in relation to each other between open and closed positions.

7. The circular stapling device of claim 6, wherein the first jaw is pivotably coupled to the second jaw.

8. The circular stapling device of claim 2, wherein the proximal portion of each of the first and second grasping members includes a grip portion.

9. The circular stapling device of claim 8, wherein the grip portion includes a loop.

10. The circular stapling device of claim 1, wherein the body of the grasping member is formed of a resilient material.

11. The circular stapling device of claim 1, further including a handle assembly, the proximal portion of the adapter assembly coupled to the handle assembly.

12. A reload assembly comprising:
a staple cartridge having an annular configuration;
a housing defining a cavity and having a proximal portion and a distal portion, the staple cartridge supported on the distal portion of the housing, the housing and the staple cartridge defining at least one channel that extends from the proximal portion of the housing through the staple cartridge; and
a grasping member movable within the at least one channel between retracted and extended positions, the grasping member including a body having a proximal portion and a distal portion, the distal portion of the body of the grasping member including a tissue engaging portion, the tissue engaging portion extending from the at least one channel to a position distal of the staple cartridge when the grasping member is in the extended position.

13. The reload assembly of claim 12, wherein the at least one channel includes a first channel and a second channel, and the grasping member includes a first grasping member and a second grasping member, the first grasping member received within the first channel and the second grasping member received within the second channel.

14. The reload assembly of claim 13, wherein the first and second channels are diametrically opposed to each other.

15. The reload assembly of claim 14, wherein the staple cartridge has an inner periphery and the first and second channels extend through the inner periphery of the staple cartridge.

16. The reload assembly of claim 13, wherein the tissue engaging portion of each of the first and second grasping members includes a hook portion.

17. The reload assembly of claim 2, wherein the tissue engaging portion of each of the first and second grasping members includes a first jaw and a second jaw that is pivotably coupled to the first jaw, the first and second jaws movable in relation to each other between open and closed positions.

18. The reload assembly of claim 13, wherein the proximal portion of each of the first and second grasping members includes a grip portion having a loop.

19. The reload assembly of claim 12, wherein the body of the grasping member is formed of a resilient material.

20. A reload assembly comprising:
a staple cartridge having an annular configuration and including an inner periphery;
a housing defining a cavity and having a proximal portion and a distal portion, the staple cartridge supported on the distal portion of the housing, the housing and the staple cartridge defining first and second diametrically disposed channels that extend from the proximal portion of the housing through the inner periphery of the staple cartridge; and
first and second grasping members movable within the first and second channels between retracted and extended positions, the first and second grasping members each including a body having a proximal portion and a distal portion, the distal portion of the body of each of the first and second grasping members including a tissue engaging portion, the tissue engaging portions extending from the first and second channels to positions distal of the staple cartridge when the first and second grasping members are in their extended positions.
